(19) United States
(12) Patent Application Publication
(10) Pub. No.: US 2022/0346697 A1
(43) Pub. Date: Nov. 3, 2022

(54) ELECTROMYOGRAPHY PROCESSING APPARATUS, ELECTROMYOGRAPHY PROCESSING METHOD AND ELECTROMYOGRAPHY PROCESSING PROGRAM

(71) Applicant: Nippon Telegraph and Telephone Corporation, Tokyo (JP)

(72) Inventors: Osamu SAISHO, Musashino-shi, Tokyo (JP); Shingo TSUKADA, Musashino-shi, Tokyo (JP); Hiroshi IMAMURA, Musashino-shi, Tokyo (JP); Atsunori MIHARA, Musashino-shi, Tokyo (JP)

(21) Appl. No.: 17/623,451

(22) PCT Filed: Jun. 28, 2019
(86) PCT No.: PCT/JP2019/025815

§ 371 Date: Dec. 28, 2021

(51) Int. Cl. A61B 5/397; A61B 5/296

(52) U.S. Cl. CPC A61B 5/397; A61B 5/296; A61B 2562/04

(57) ABSTRACT

An electromyography processing apparatus 1 includes an electromyography acquiring unit 21 configured to generate electromyography data 11 indicating the time course of an electromyography acquired from an electrode 2 set on each of left and right muscles which are paired of an exerciser performing an exercise in which the left and right muscles are alternately used, and an evaluation unit 23 configured to calculate and output a switching index indicating that left and right muscles are alternately used, from an electromyography of the left muscle and an electromyography of the right muscle acquired at an identical time.

### TECHNICAL FIELD

The present disclosure relates to an electromyography processing apparatus, an electromyography processing method, and an electromyography processing program.

### BACKGROUND ART

Electromyography is physiological information that directly represents how to use the body, and in order to improve various sports skills, the utilization of electromyography has attracted attention. Electromyography is a voltage that occurs when a muscle is moved. Electromyography is also referred to as electromyography (EMG). The amplitude of electromyography increases when strength is applied, and approaches 0 when strength is lost. It is expected that by focusing on electromyography, an exerciser himself/herself will be able to interpret whether the muscles are properly used at a training site and apply this to the training to improve his/her performance.

However, electromyography is only an electrical signal, and thus it is difficult to interpret electromyography data, and there is a need for a technique for processing electromyography data such that an exerciser himself/herself can understand the electromyography data. For example, there is a technique in which, for a plurality of muscles, the timing at which a muscle moves and electromyography increases is detected and a sound at a frequency applied to each muscle is generated to provide feedback to an exerciser by means of the sound (see NPL 1).

### CITATION LIST

### Non Patent Literature

NPL 1: NTT Communication Science Laboratories, “Open House 2016, Shaping the Athletic Brain!”, [online], 2016, NTT, [Searched on Jun. 20, 2019]; Internet (URL: http://www.kecl.ntt.co.jp/openhouse/2016/exhibition/28/index.html)

### SUMMARY OF THE INVENTION

### Technical Problem

In an exercise such as running or pedaling a bike, left and right muscles of the body that are paired are used alternately. It is important that the left and right muscles move with little impact on each other. For example, there is a method in which a power meter is mounted on each bike pedal to confirm that there is no difference between the strength applied to the left pedal and the strength applied to the right pedal. However, this method cannot identify a factor for their being a difference between the strengths applied to the left and right pedals. There is no method for evaluating the impact of each muscle in an exercise in which left and right muscles are used alternately.

The present disclosure has been made in view of the above circumstances, and an object of the present disclosure is to provide a technique for evaluating the impact of each muscle in an exercise in which left and right muscles that are paired are used alternately.

### Means for Solving the Problem

An electromyography processing apparatus according to one aspect of the present disclosure includes: an electromyography acquiring unit configured to generate electromyography data indicating a time course of an electromyography acquired from an electrode set on a left muscle of an exerciser and a time course of an electromyography acquired from an electrode set on a right muscle of the exerciser, the left muscle and the right muscle being paired, and the exerciser performing an exercise in which the left muscle and the right muscle are alternately used; and an evaluation unit configured to calculate and output, from an electromyography of the left muscle and an electromyography of the right muscle both acquired at an identical time, a switching index indicating that the left muscle and the right muscle are alternately used, the left muscle and the right muscle being paired.

An electromyography processing method according to one aspect of the present disclosure includes: generating, by a computer, electromyography data indicating a time course of an electromyography acquired from an electrode set on a left muscle of an exerciser and a time course of an electromyography acquired from an electrode set on a right muscle of the exerciser, the left muscle and the right muscle being paired, and the exerciser performing an exercise in which the left muscle and the right muscle are alternately used; and calculating and outputting, by the computer, from an electromyography of the left muscle and an electromyography of the right muscle both acquired at an identical time, a switching index indicating that the left muscle and the right muscle are alternately used, the left muscle and the right muscle being paired.

An aspect of the present disclosure is an electromyography processing program causing a computer to operate as the electromyography processing apparatus.

### Effects of the Invention

According to the present disclosure, it is possible to provide a technique for evaluating the impact of each muscle in an exercise in which left and right muscles that are paired are alternately used.

### BRIEF DESCRIPTION OF DRAWINGS

FIG. 1 is a diagram for explaining functional blocks of an electromyography processing apparatus according to an embodiment of the present disclosure.

FIG. 2 is a diagram for explaining an example of tights in which electrodes are provided.

FIG. 3 is a flowchart for explaining preprocessing by a preprocessing unit.

FIG. 4 shows an example of signal input and output by the preprocessing unit.

FIG. 5 is a diagram for explaining a root-mean-square calculated by the preprocessing unit.

FIG. 6 is a flowchart for explaining evaluation processing by an evaluation unit.

FIG. 7 is an example of a switching index calculated based on an electromyography.

FIG. 8 is an example of output from the evaluation unit.

FIG. 9 is a diagram for explaining a hardware configuration of a computer.

### DESCRIPTION OF EMBODIMENTS

Hereinafter, an embodiment of the present disclosure will be described with reference to the drawings. Note that in descriptions of the drawings, the same components are denoted by the same reference signs and explanations thereof will be omitted.

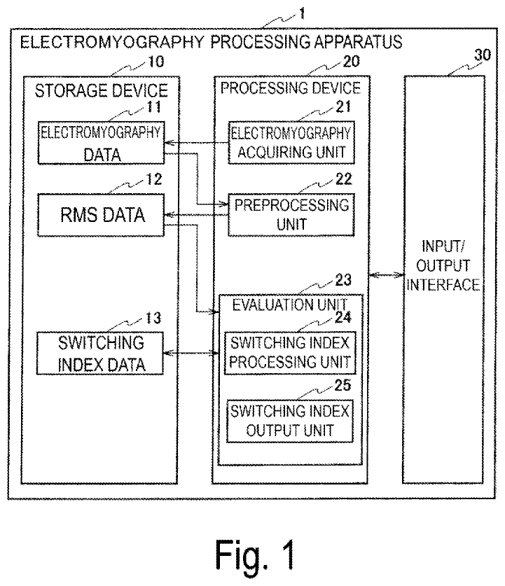

An electromyography processing apparatus 1 according to the embodiment of the present disclosure will be described with reference to FIG. 1. The electromyography processing apparatus 1 outputs data with which an exerciser performing an exercise such as a cycling competition or running in which repetitions are repeated can grasp changes in muscle movement during the repetitive exercise. In the embodiment of the present disclosure, in particular, the exerciser performs an exercise in which left and right muscles that are paired are alternately used.

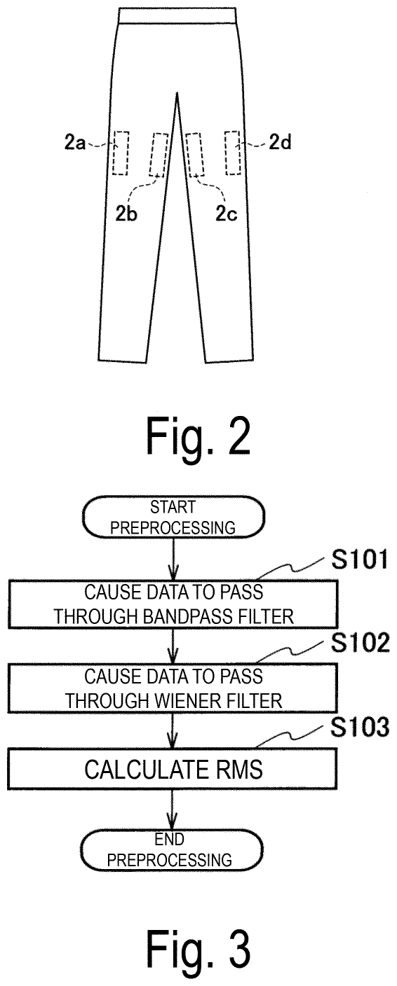

On the inside of an item of clothing worn by the exerciser, electrodes 2a to 2d are provided as illustrated in FIG. 2, and the electrodes 2a to 2d come into contact with the skin of the exerciser. The electromyography processing apparatus 1 acquires, via the electrodes 2a to 2d, electromyographies of muscles located subcutaneously at the locations where the electrodes are provided. The electrodes 2a to 2d may be attached to the skin of the exerciser.

In the embodiment of the present disclosure, the electrodes are provided on pairs of left and right muscles. In the example illustrated in FIG. 2, the electrodes 2a and 2d acquire electromyographies of the left and right vastus lateralis muscles, respectively. The electrodes 2b and 2c acquire electromyographies of the left and right biceps femoris muscles (hamstrings), respectively. During the exercise by the exerciser, the electromyography processing apparatus 1 sequentially acquires electromyographies obtained from the electrodes, analyzes the acquired electromyographies, and outputs the result of the analysis. Note that when it is not particularly necessary to differentiate between the electrodes 2a to 2d, they may be referred to as the electrodes 2. Note that the positions and the number of the electrodes 2 illustrated in FIG. 2 are exemplary and without limitation. The electrodes 2 are provided at positions at which it is possible to acquire the electromyography of a muscle set to be measured, as appropriate.

As illustrated in FIG. 1, the electromyography processing apparatus 1 according to the embodiment of the present disclosure includes a storage device 10 and a processing device 20.

The storage device 10 stores an electromyography processing program and stores electromyography data 11, RMS data 12, and switching index data 13.

The electromyography data 11 is data indicating the time course of an electromyography acquired from an electrode set on each of left and right muscles that are paired of an exerciser performing an exercise in which the left and right muscles are used alternately. The electromyography data 11 is data in which a value of the electromyography obtained from the electrodes 2 is associated with the time at which the value is acquired. When electromyographies are acquired from a plurality of muscles, the electromyography data 11 is generated for each muscle.

The RMS data 12 includes a root-mean-square (RMS) value of electromyographies for each predetermined time. The RMS data 12 is data in which a calculated RMS value of electromyographies is associated with a time corresponding to the RMS value. When the electromyography data 11 includes electromyographies of a plurality of muscles, the RMS data 12 is generated for each muscle.

The switching index data 13 includes a switching index calculated for each predetermined time. The switching index data 13 is data in which a calculated switching index is associated with an identifier of a time corresponding to the switching index. The switching index data 13 may be generated for each of the left and right muscles which are paired, or may be generated for each of the left and right muscle groups by setting each of a left muscle group and a right muscle group as a block.

The processing device 20 includes an electromyography acquiring unit 21, a preprocessing unit 22, and an evaluation unit 23.

The electromyography acquiring unit 21 generates the electromyography data 11 indicating the time course of an electromyography acquired from the electrodes 2 set on each of left and right muscles that are paired of an exerciser performing an exercise in which left and right muscles are alternately used. The electromyography acquiring unit 21 generates the electromyography data 11 for each muscle corresponding to each electrode. In the embodiment of the present disclosure, the electromyography acquiring unit 21 sequentially acquires an electromyography from the electrodes 2 set on each of the left and right muscles of the exerciser performing the exercise in which the left and right muscles are alternately used.

The preprocessing unit 22 removes noise from an electromyography value of the electromyography data 11 and calculates an RMS value on the basis of the electromyography value after noise removal to generate the RMS data 12. The preprocessing unit 22 calculates an RMS value of the electromyography data 11 for each predetermined time to generate root-mean-square square data (RMS data 12) including an RMS value for each time. When the electromyographies of a plurality of muscles are acquired, the preprocessing unit 22 generates the RMS data 12 for each of the plurality of muscles.

Preprocessing by the preprocessing unit 22 will be described with reference to FIG. 3.

First, in step S101, the preprocessing unit 22 causes the electromyography data 11 to pass through a bandpass filter. In step S102, the preprocessing unit 22 causes the data that has passed through the bandpass filter in step S101 to pass through a Wiener filter.

In step S103, the preprocessing unit 22 calculates a root-mean-square for the data that has passed through the Wiener filter in step S102 to generate the RMS data 12.

The preprocessing unit 22 causes the electromyography data 11 to pass through the bandpass filter to filter out frequencies other than the frequency of an electromyography. The electromyography data 11 including an electromyography acquired from the electrodes 2 includes various noise such as noise generated by a body movement called a “motion artifact”, and noise generated by electricity or the like occurring in the skin even without movement. When the electromyography data 11 passes through the bandpass filter, noise outside the frequency band of an electromyography is removed. As a result, the electromyography data 11 can be narrowed into the frequency band of the electromyography to be acquired.

The frequency of the bandpass filter is set in accordance with the noise included in the electromyography data 11. The preprocessing unit 22 is not limited to a bandpass filter that defines an upper limit value and a lower limit value, and a high-pass filter or a low-pass filter may be used which does not define either the upper limit or the lower limit. The upper limit value and the lower limit value of the bandpass filter are determined on the basis of a sampling frequency of the electromyography to be acquired or a characteristic of a device. For example, in a case where the sampling frequency is 500 Hz, the upper limit value is set to 249 Hz on the basis of a sampling theorem, and the lower limit is set to 10 Hz from a main frequency characteristic of the electromyography. As a frequency filtering method, for example, a Butterworth filter is common, but the frequency filtering method is not limited thereto.

The preprocessing unit 22 applies the Wiener filter to the data that has passed through the bandpass filter to remove noise on the entire electromyography data 11, thereby removing signals (noise) other than an electrical signal generated by muscle activation. When data has been acquired for measuring noise intensity, the intensity of noise removal by the Wiener filter is determined on the basis of the data. When the noise intensity has not been measured, the intensity of noise removal is determined on the basis of the electromyography data 11. The preprocessing unit 22 determines the intensity of noise removal on the basis of, for example, electromyographies of all sections (each time) of the electromyography data 11.

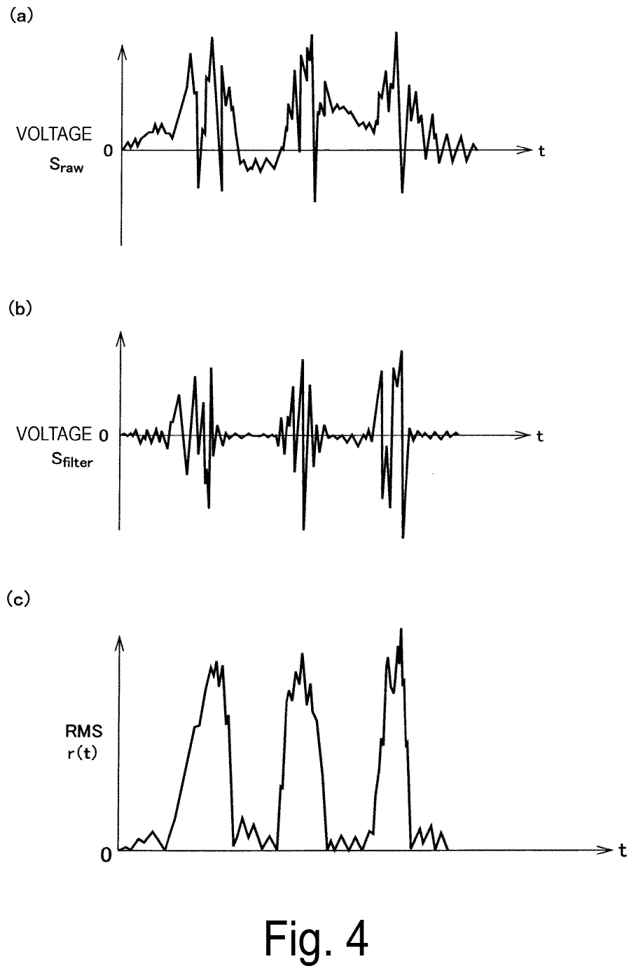

When the preprocessing unit 22 applies the bandpass filter and the Wiener filter to the electromyography data 11 shown in FIG. 4(a) to remove noise, the data shown in FIG. 4(b) is obtained. In the data shown in FIG. 4(b), it is easier to distinguish between a section in which the voltage is close to 0 and a section in which the voltage is not 0, than in the data shown in FIG. 4(a).

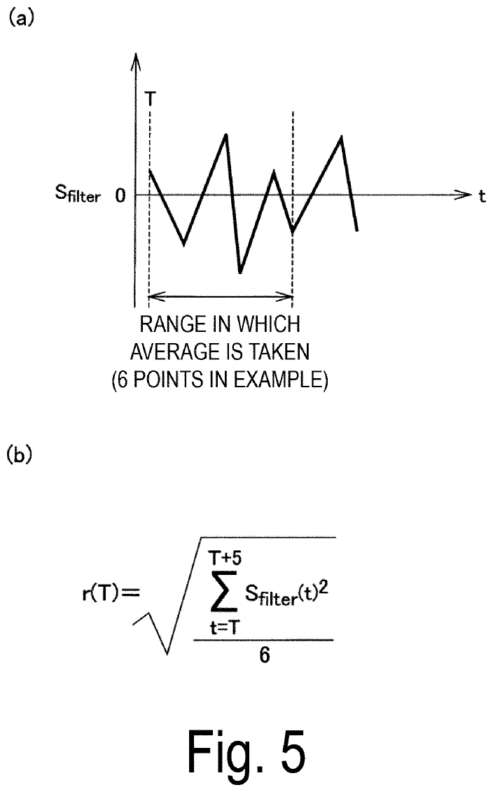

In addition, the preprocessing unit 22 calculates a root-mean-square on the data that has passed through the bandpass filter and the Wiener filter. As shown in FIG. 5(a), the preprocessing unit 22 calculates r(T), which is the RMS value, by the equation shown in FIG. 5(b) on data in a range for which an average is taken, out of the data that has passed through the filters. The preprocessing unit 22 repeats the processing of calculating a root-mean-square for each section to generate the RMS data 12.

As a result, the preprocessing unit 22 obtains the data shown in FIG. 4(c). Compared to FIG. 4(b), the signal shown in FIG. 4(c) can express the output of the electromyography in a single motion as one unit.

The evaluation unit 23 refers to the RMS data 12, and calculates and outputs an index that quantifies the exercise performed by the exerciser. The evaluation unit 23 includes a switching index processing unit 24 and a switching index output unit 25.

The switching index processing unit 24 calculates a switching index indicating that left and right muscles that are paired are alternately used, from an electromyography of the left muscle and an electromyography of the right muscle acquired at an identical time.

First, the switching index processing unit 24 normalizes the RMS data 12. The RMS data 12 includes the RMS value of the electromyographies for each predetermined time. The electromyography varies greatly depending on the manner of sweating, the position of the electrodes with respect to the muscles, the intensity of the exercise, and the like. The switching index processing unit 24 uses a value obtained by normalizing the RMS value in a sliding window to calculate a switching index in order to suppress impact from the manner of sweating, the positions of the electrodes with respect to the muscles, the intensity of the exercise, and the like. The window width of the sliding window is a time a, which can be recognized as one block of the exercise. In the embodiment of the present disclosure, the time a of the window width is 4 seconds. The step width of the sliding window is the predetermined time for which the RMS value is calculated in the RMS data 12.

In the sliding window set in this manner, the RMS value normalized by means of Equation (1) is referred to as a normalized RMS value in the present embodiment of the present disclosure.

$$\begin{matrix}[ Math . 1 ] & \\ {\tilde{r}}_{t} = \frac{{r}_{t} - {min}_{s ∈ [ t - \frac{a}{2} , t + \frac{a}{2} ]} {r}_{s}}{{max}_{s ∈ [ t - \frac{a}{2} , t + \frac{a}{2} ]} {r}_{s} - {min}_{s ∈ [ t - \frac{a}{2} , t + \frac{a}{2} ]} {r}_{s}} & Equation ( 1 )\end{matrix}$$

$${r}_{t} : RMS value at time t$$

$${\tilde{r}}_{t} : Normalized RMS value at time t$$

$$a : Window width$$

The normalized RMS value falls within a range of [0, 1]. RMS value normalization is performed for each RMS value of the RMS data 12.

When outputting the switching index for left and right muscles which are paired, the switching index processing unit 24 normalizes the RMS value for each of the left and right muscles. The switching index processing unit 24 outputs the switching index using the normalized RMS value for the left muscle and the normalized RMS value for the right muscle. The left and right muscles are, for example, left and right biceps femoris muscles, or left and right vastus lateralis muscles.

When outputting the switching index for left and right muscle groups which are paired, the switching index processing unit 24 calculates the normalized RMS value for each muscle. The switching index processing unit 24 calculates an average value of the normalized RMS values for the left muscle group for a predetermined time and an average value of the normalized RMS values for the right muscle group for the same predetermined time, and outputs the switching index from the average values for the left and right muscle groups for the same predetermined time. The left and right muscle groups which are paired are, for example, the three pairs of left and right muscle groups of the left and right vastus lateralis muscles, rectus femoris muscles, and vastus medialis muscles, or the two pairs of left and right muscle groups of the left and right biceps femoris muscles and gluteus maximus muscles.

The switching index processing unit 24 calculates the switching index from multiplication of the electromyographies of the left and right muscles acquired at an identical time. In the embodiment of the present disclosure, a case where the switching index is calculated from the multiplication of the values obtained by normalizing the RMS value of the electromyography is described, but the method for calculating the switching index is not limited thereto. The switching index may be calculated from multiplication of the electromyographies themselves. It is preferable that there be no difference in the fluctuation range of the electromyography between the left and right muscles.

In the embodiment of the present disclosure, the switching index processing unit 24 calculates the switching index from the multiplication of the values obtained by normalizing the electromyographies of the left and right muscles acquired at an identical time. The values obtained by normalizing the electromyographies each correspond to a value obtained by normalizing the RMS value of the electromyography. The value obtained by normalizing the RMS value of the electromyography is used, and thus the switching index processing unit 24 can suppress impact from an instantaneous change in the electromyography and a difference in the fluctuation range of the electromyography between the left and right muscles, to numerically represent the switching of the left and right muscles.

The switching index processing unit 24 calculates the switching index by means of Equation (2).

[Math. 2]

eₜ=\tilde{r}_{l,t}\tilde{r}_{r,t} Equation (2)

eₜ: Switching index at time t
\tilde{r}_{l,t}: Normalized RMS value of left muscle at time t
\tilde{r}_{r,t}: Normalized RMS value of right muscle at time t

The RMS value obtained by normalizing the electromyography of each muscle is in the range of [0, 1], and thus the switching index is also in the range of [0, 1]. The switching index being close to 0 means that at least one of the values is close to 0 and that even when strength is applied with one of the muscles, no strength is applied with the other muscle. When a situation in which the switching index is close to 0 continues, strength is alternately applied with the left and right muscles which are paired, and the left and right muscles are successfully switched.

The switching index being close to 1 means that both left and right values are close to 1 and that when strength is applied with one of the muscles, strength is also applied with the other muscle. When a situation in which the switching index is close to 1 continues, it is considered that the left and right muscles which are paired are not being successfully switched, that strength is being applied with both the left and right muscles simultaneously, and that the power of the left muscle and the power of the right muscle are canceling out each other.

The switching index output unit 25 outputs the switching index output by the switching index processing unit 24. The switching index output unit 25 may display the switching index at each time in a time-series graph. The switching index output unit 25 may output a result obtained by converting the switching index by means of a predetermined conversion, rather than the switching index itself. The switching index output unit 25 may represent the switching index by the number of points out of 100 points such that the switching index “0” is represented by 100 points. The switching index output unit 25 may represent the switching index by a graded scale such as “Good”, “Average”, and “Bad” such that the switching index 0 is represented by “Good”.

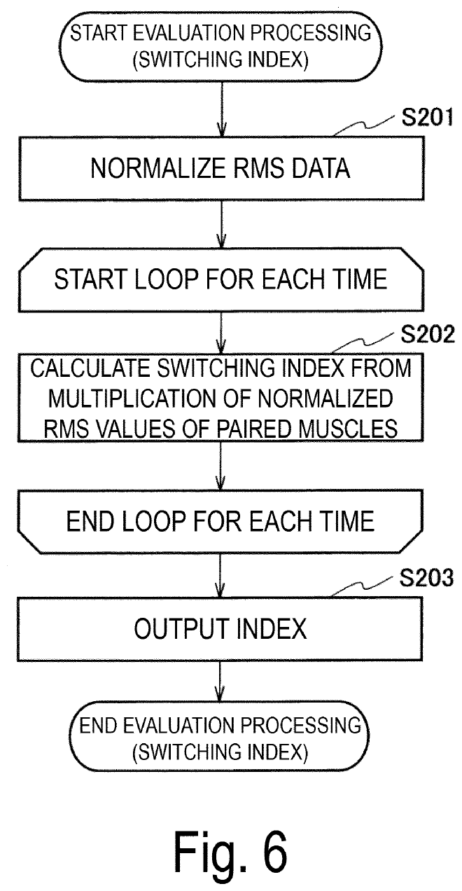

Evaluation processing by the evaluation unit 23 according to the embodiment of the present disclosure will be described with reference to FIG. 6.

First, in step S201, the evaluation unit 23 normalizes the RMS data 12 of each muscle by means of Equation (1).

The evaluation unit 23 performs processing in step S202 for a normalized value at each time. In step S202, the evaluation unit 23 calculates, by means of Equation (2), a switching index at a predetermined time from the multiplication of normalized RMS values of paired left and right muscles for this time.

In step S203, the evaluation unit 23 outputs the switching index calculated in step S202.

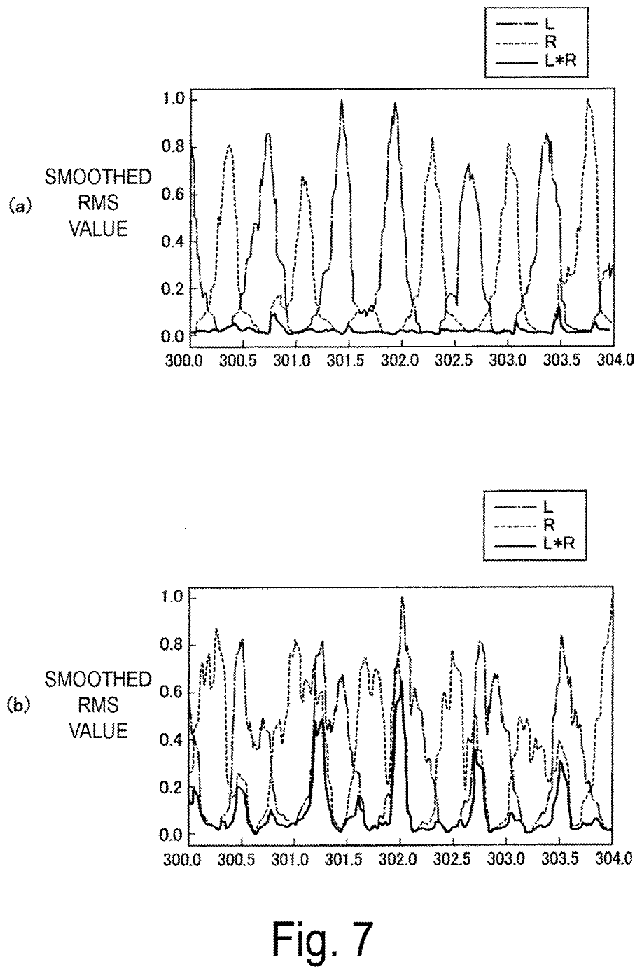

FIG. 7 shows an example of the switching index output by the evaluation unit 23. FIG. 7 shows changes in the values obtained by normalizing the RMS values of the electromyographies and the switching index for the biceps femoris muscles when a bike is pedaled under the same conditions. FIG. 7(a) shows data of a professional sportsperson and FIG. 7(b) shows data of an amateur sportsperson. In FIG. 7, the solid line indicates a switching index. The dot-dash line and dotted line indicate normalized RMS values of a pair of muscles. The dot-dash line indicates the value for the left muscle, and the dotted line indicates the value for the right muscle.

In a cycling competition, the stepping operation alternates from side to side during pedaling. The electromyography of each of the left and right biceps femoris muscles increases each time stepping is performed. While stepping on the left and stepping on the right are repeated, it is ideal for peaks of values for electromyographies of the left and peaks of values for electromyographies of the right to appear alternately.

In the data of the professional sportsperson shown in FIG. 7(a), both the peaks of values for the left muscle and the peaks of values for the right muscle are sharp. In addition, the respective peaks alternate regularly. When one of the right biceps femoris muscle and the left biceps femoris muscles is activated, the other thereof is not activated. The switching index maintains a value close to 0, indicating that the left and right muscles are being switched successfully.

In the data of the amateur sportsperson shown in FIG. 7(b), both the peaks of values for the left muscle and the peaks of values for the right muscle are not sharp. In addition, the intervals between the peaks are narrow. Both the left and right muscles are activated for a long time, and with such a time the switching index value becomes a large value. The switching index often has a value that deviates from 0 compared to that of the professional sportsperson, indicating that the left and right muscles are not switched successfully. While the left and right muscles should be alternately activated, when both the left and right muscles are activated, it is thought that the movement of one of the left and right muscles cancels out a force such as the driving force generated by the other thereof and vice versa.

The electromyography processing apparatus 1 according to the embodiment of the present disclosure can quantitatively represent, as the switching index, that bilaterally symmetrical muscles are alternately used and that the respective movements do not obstruct each other. The electromyography processing apparatus 1 can show an exerciser the efficiency of switching between left and right by the exerciser.

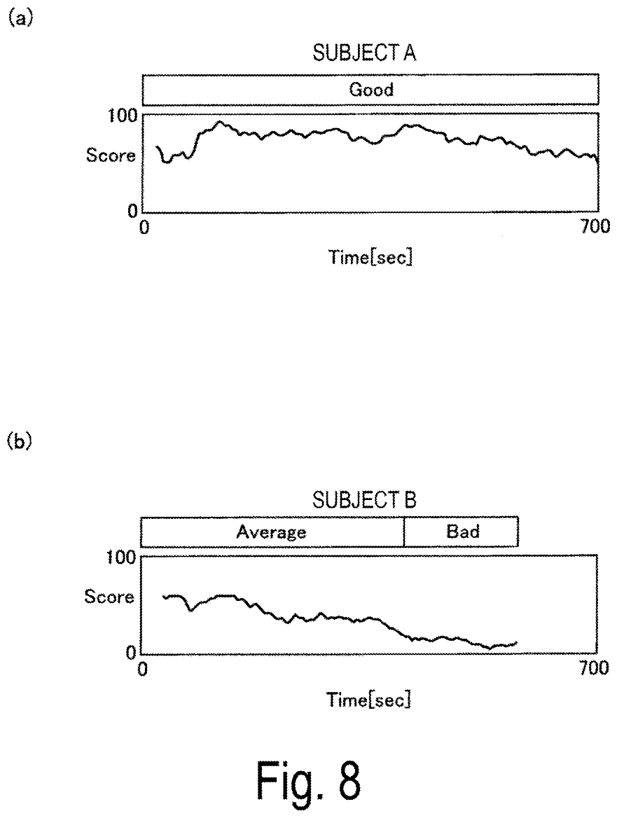

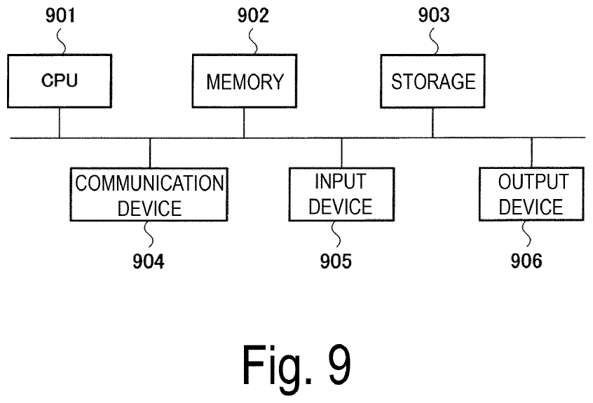

An example of an evaluation output by the evaluation unit 23 will be described with reference to FIG. 8. FIG. 8 shows a score at each time calculated from the switching index. When the switching index is closer to 0, the score approaches 100, and when the switching index is closer to 1, the score approaches 0. Furthermore, in FIG. 8, three evaluations of “Good”, “Average”, and “Bad” are associated in accordance with the transition of the score. FIG. 8(a) shows data of a professional sportsperson and FIG. 8(b) shows data of an amateur sportsperson.

In FIG. 8(a), a state in which the score is close to 100 is maintained and overall an evaluation of “Good” is given. In FIG. 8(b), the score is low compared to that in FIG. 8(a), and an evaluation of “Average” is initially given, but an evaluation of “Bad” is given in the later half in which the score further lowers.

The electromyography processing apparatus 1 according to the embodiment can output the switching index that evaluates the impact of each muscle in an exercise in which left and right muscles that are paired are alternately used on the basis of the electromyographies measured simultaneously from the left and right muscles.

As the electromyography processing apparatus 1 according to the present embodiment described above, for example, a general-purpose computer system including a central processing unit (CPU; processor) 901, a memory 902, a storage 903 (hard disk drive (HDD) or a solid state drive (SSD)), a communication device 904, an input device 905, and an output device 906 is used. The CPU 901 is the processing device 20. The memory 902 and the storage 903 are the storage device 10. In the computer system, the CPU 901 executes the electromyography processing program loaded into the memory 902 to implement each function of the electromyography processing apparatus 1.

Note that the electromyography processing apparatus 1 may be implemented by one computer or may be implemented by a plurality of computers. The electromyography processing apparatus 1 may be a virtual machine implemented on a computer.

The electromyography processing program may be stored in a computer-readable recording medium such as an HDD, an SSD, a universal serial bus (USB) memory, a compact disc (CD), or a digital versatile disc (DVD), or may be distributed through a network.

The present disclosure is not limited to the embodiment, and various modifications can be made within the scope of the gist of the present disclosure.

### REFERENCE SIGNS LIST

1 Electromyography processing apparatus

10 Storage device

11 Electromyography data

12 RMS data

13 Switching index data

20 Processing device

21 Electromyography acquiring unit

22 Preprocessing unit

23 Evaluation unit

24 Switching index processing unit

25 Switching index output unit

30 Input/output interface

901 CPU

902 Memory

903 Storage

904 Communication device

905 Input device

906 Output device

1. An electromyography processing apparatus comprising:
an electromyography acquiring unit comprising one or more hardware processors and configured to generate electromyography data indicating a time course of an electromyography acquired from an electrode set on a left muscle of an exerciser and a time course of an electromyography acquired from an electrode set on a right muscle of the exerciser, the left muscle and the right muscle being paired, and the exerciser performing an exercise in which the left muscle and the right muscle are alternately used; and
an evaluation unit comprising the one or more hardware processors and configured to calculate and output, from an electromyography of the left muscle and an electromyography of the right muscle both acquired at an identical time, a switching index indicating that the left muscle and the right muscle are alternately used, the left muscle and the right muscle being paired.

2. The electromyography processing apparatus according to claim 1, wherein the evaluation unit configured to calculate the switching index from multiplication of the electromyography of the left muscle and the electromyography of the right muscle both acquired at the identical time.

3. The electromyography processing apparatus according to claim 1, wherein the evaluation unit configured to calculate the switching index from multiplication of values obtained by normalizing each of the electromyography of the left muscle and the electromyography of the right muscle both acquired at the identical time.

4. An electromyography processing method comprising:
generating, by a computer, electromyography data indicating a time course of an electromyography acquired from an electrode set on a left muscle of an exerciser and a time course of an electromyography acquired from an electrode set on a right muscle of the exerciser, the left muscle and the right muscle being paired, and the exerciser performing an exercise in which the left muscle and the right muscle are alternately used; and
calculating and outputting, by the computer, from an electromyography of the left muscle and an electromyography of the right muscle both acquired at an identical time, a switching index indicating that the left muscle and the right muscle are alternately used, the left muscle and the right muscle being paired.

5. The electromyography processing method according to claim 4, wherein in the calculating and outputting, the switching index is calculated from multiplication of the electromyography of the left muscle and the electromyography of the right muscle both acquired at the identical time.

6. The electromyography processing method according to claim 4, wherein in the calculating and outputting, the switching index is calculated from multiplication of values obtained by normalizing each of the electromyography of the left muscle and the electromyography of the right muscle both acquired at the identical time.

7. A computer-readable recording medium storing [[An]] an electromyography processing program executable to cause one or more computers to perform operations comprising:
generating, by a computer, electromyography data indicating a time course of an electromyography acquired from an electrode set on a left muscle of an exerciser and a time course of an electromyography acquired from an electrode set on a right muscle of the exerciser, the left muscle and the right muscle being paired, and the exerciser performing an exercise in which the left muscle and the right muscle are alternately used; and
calculating and outputting, by the computer, from an electromyography of the left muscle and an electromyography of the right muscle both acquired at an identical time, a switching index indicating that the left muscle and the right muscle are alternately used, the left muscle and the right muscle being paired.
